(19) United States
(12) Patent Application Publication
(10) Pub. No.: US 2019/0070097 A1
(43) Pub. Date: Mar. 7, 2019

(54) NAIL COMPOSITION

(71) Applicant: Slixir Ltd, Castleford (GB)

(72) Inventor: Helen GILMORE, Castleford (GB)

(21) Appl. No.: 16/090,100

(22) PCT Filed: Mar. 27, 2017
(86) PCT No.: PCT/GB2017/050857

§ 371 Date: Sep. 28, 2018

(30) Foreign Application Priority Data
Mar. 29, 2016 (GB) 1605257.3

(51) Int. Cl. A61K 8/92; A61Q 3/02; A61K 8/9783; A61K 8/06

(52) U.S. Cl. CPC A61K 8/922; A61K 8/062; A61K 8/9783; A61Q 3/02

(57) ABSTRACT

Certain aspects of the present invention relate to a composition for application to a cured, applied polymeric nail coating, hands and cuticles. Particularly, although not exclusively certain embodiments of the present invention relate to a composition which acts to prolong duration and/or maintain the appearance of an applied nail coating applied to a nail. Aptly, the composition may act as a post-application plasticizer of the nail coating.

### FIELD OF THE INVENTION

Certain aspects of the present invention relate to a composition for application to a cured, applied polymeric nail coating, hands and cuticles. Particularly, although not exclusively certain embodiments of the present invention relate to a composition which acts to prolong duration and/or maintain the appearance of an applied nail coating applied to a nail. Aptly, the composition may act as a post-application plasticizer of the nail coating.

### BACKGROUND TO THE INVENTION

Nail polish wearers traditionally seek to extend the wear of their nail coating for as long as possible. Currently, it has been the nail coating itself, top and basecoats which have been marketed to consumers as promising increased longevity and effective manicure maintenance.

Base and top coats, proper nail preparation (to avoid chipping or flaking of the natural nail beneath) and advancements in formulation are all know as ways to improve the life of nail coatings. Longer-wear compositions such as so-called “gel polish” became available in the UK around 2005. These compositions are a hybrid between traditional nail polish compositions and more invasive acrylic or gel nail ‘extension’ services. They typically offer up to a two week nail coating life span. Typically the gel polish compositions are photo-crosslinkable and therefore can be cured under UV or LED light.

Use has already been made in polish formulation of plant oils as plasticizers in a formula. As the nail coating ages, the plasticizers (whether they be plant or other chemicals such as dibutyl phthalate) and solvents are lost to evaporation and contact with other external chemicals such as household cleaning products and soaps. The loss of plasticizer is often accelerated by decomposition of nitrocellulose which produces nitrous oxides which in turn combine with water to form nitrous and nitric acids. Such acids typically affect the stability of plasticizers and their interaction with the polymeric material of the nail coating, leading to premature degradation of the coating.

It is an aim of certain embodiments of the present invention to at least partially mitigate the problems associated with the prior art.

It is an aim of certain embodiments of the present invention to provide a composition which acts to continue to plasticize and therefore prolong the duration and/or appearance of a nail product which is applied to a person's nail.

### DESCRIPTION OF CERTAIN EMBODIMENTS OF THE INVENTION

In a first aspect, there is provided a composition, the composition being for application to an applied, cured nail coating, and wherein the composition comprises at least one aqueous phase and at least one oily phase, the oily phase comprising at least one wax ester and at least one plant oil component, wherein the composition is in the form of a cream. Aptly the the at least one plant oil component has a positive ionic charge.

In certain embodiments, the composition is considered to mimic the effects of the original plasticizer present in the applied formula. Furthermore, it is considered that the composition acts to replace the original plasticizer which is lost to solvent evaporation and water transpiration. In certain embodiments, the plant oils in the composition render the nail coating more hydrophobic, effectively creating an occlusive layer which slows down solvent evaporation, helps to prevent water absorption and promotes flexibility.

Aptly, the composition is in the form of an oil-in-water composition.

Aptly, the composition is a cream. As used herein, the term “cream” refers to a composition which has one or more of the following characteristics:
a. Forms a stiff peak (as opposed to a soft peak or no peak, as a lotion would be);
b. Opaque;
c. Dries almost immediately on application;
d. Semi-solid;
e. Non-pourable;
f. High viscosity; and
g. Low loss of mass on drying (approximately 30-40% loss of mass as compared to the initial mass of the composition on application).

In certain embodiments, the composition comprises greater than 20% w/w water and volatile components and less than 50% w/w hydrocarbons, waxes or polyols.

In certain embodiments, the at least one plant oil component comprises a fatty acid selected from oleic acid, palmitic acid, linoleic acid and/or combinations thereof.

Aptly, the at least one plant oil component is avocado oil or a derivative thereof.

In certain embodiments, the composition comprises the at least one plant oil component in a concentration of between about 0.1 and about 2.5% w/w.

In certain embodiments, the composition comprises the at least one plant oil component in a concentration of between about 0.1 and about 0.5% w/w.

In certain embodiments, the composition comprises the at least one plant oil component in a concentration of between about 0.1 and about 0.3% w/w. e.g. about 0.2% w/w.

Aptly, the at least one wax ester is a jojoba oil wax ester. The composition may comprise jojoba oil and/or a hydrogenated jojoba oil. In certain embodiments, the wax ester in a concentration of between about 1% and about 3% w/w of the total weight of the composition

Aptly, wax esters are esters of long-chain fatty acids and long-chain fatty alcohols. Aptly, the jojoba wax ester comprises long chain fatty alcohols esterified with long chain (C35 to C46) fatty acids.

In certain embodiments, the composition does not comprise an animal or mineral oil, or wax ester thereof.

In certain embodiments, the aqueous phase comprises deionised water, and wherein optionally the deionised water is provided in a concentration of between 50% and 75% w/w of the composition.

In certain embodiments, the composition further comprises at least one emulsifier. Aptly, the emulsifier is a non-ionic emulsifier, wherein optionally the non-ionic emulsifier comprises glyceryl stearate and PEG-100 stearate. Aptly, the composition comprises the emulsifier in a concentration of between about 1% and 5% w/w. Aptly, the emulsifier is provided in a concentration of about 2% w/w.

In certain embodiments, the composition further comprises a perfume.

In certain embodiments, the composition further comprises a texturizing agent. In certain embodiments, the texturizing agent comprises a plurality of particles.

In certain embodiments, the composition further comprises at least one preservative.

In certain embodiments, the composition does not contain an alcohol.

In certain embodiments, the composition further comprises at least one anti-inflammatory agent. In certain embodiments, the anti-inflammatory agent is a metal component. Aptly, the metal component is selected from a silver-containing component, a zinc containing component and a copper containing component. Aptly, the copper containing component is a copper complex.

In certain embodiments, the composition comprises a plurality of anti-inflammatory agents. Aptly, the composition further comprises colloidal oat flour.

In certain embodiments, the composition comprises one or more active agents such as for example an anti-inflammatory agent. Anti-inflammatory agents may be used to heal damaged nail cuticles and/or skin surrounding the nail.

In certain embodiments, the anti-inflammatory agent is a metal compound such as for example a compound comprising silver, zinc and/or copper. In certain embodiments, the composition comprises a copper compound e.g. Acqua-Biomin Copper Y3 PF (available from Lonza). Copper is an anti-inflammatory and therefore in certain embodiments the composition may be used to heal damaged cuticles or other skin surrounding the nail.

In certain embodiments, the composition further comprises colloidal oat flour. Aptly, the colloidal oat flour acts as an anti-inflammatory and antioxidant. Aptly, the colloidal oat flour comprises one or more of beta-glucans, phenols, starch, savenacins, saponins, flavonoids and Vitamin E.

In certain embodiments, the composition further comprises a chelating agent, wherein optionally the chelating agent is EDTA or a salt thereof.

In certain embodiments, the composition further comprises at least one emollient.

In certain embodiments, the composition does not comprise hydrophobic silica airgel particles, such as aerogel silylated silica particles,

In certain embodiments, the composition consists essentially of,
at least one aqueous phase consisting essentially of, deionised water,
at least one oily phase consisting essentially of avocado oil and a jojoba oil wax ester;
EDTA or a salt thereof;
a non-ionic emulsifier;
at least one copper compound;
at least one preservative;
a texturizing agent; and
colloidal oat flour.

Aptly, the nail coating is applied to a nail. Aptly, the nail may be a person's nail and/or a false nail which it itself has been adhered to a person's nail. Aptly, the nail coating is cured either by ambient heat or artificial UV light after being applied to a nail.

In certain embodiments, the composition maintains and/or enhances the visual appearance of the nail coating.

In certain embodiments, the composition is adapted to prevent and/or reduce evaporation of solvents from already applied nail coatings.

In certain embodiments, the composition is adapted to replace plasticizing oils in already cured nail coatings.

In certain embodiments, the composition is adapted to reduce and/or prevent brittleness in nail coatings. Brittleness is considered to be one of the principal causes of nail coating degradation and therefore the composition may have utility in preventing and/or reducing degradation of a nail coating, e.g. a nail coating which is applied to a nail.

In certain embodiments, the composition may extend the duration that the nail coating is adhered to a person's nail. Aptly, the duration is extended as compared to the duration the nail coating is adhered to a human's nail without application of the composition described herein.

In a further aspect of the present invention, there is provided a method of extending a duration of adherence of a nail coating to a nail, comprising the step of applying a composition as described herein to the nail coating. Aptly, the nail may be a human's nail and/or a false nail which has itself has been adhered to a person's nail.

In certain embodiments, the nail coating is a nail varnish. Nail varnish is typically based on nitrocellulose which is dissolved in an organic solvent.

In certain embodiments, the nail coating is an acrylic-based composition. In certain embodiments, the nail coating is a gel nail coating. In certain embodiments, the nail coating is a photo-polymerizable and/or photo-crosslinkable composition. Such compositions are described in, for example, U.S. Pat. No. 7,375,144 and WO2012/130604.

In certain embodiments, the nail coating comprises a compound provided in one or more of the following compound classes:
1. Acrylate;
2. Methacrylate;
3. Cyanoacrylate;
4. Urethane Acrylate; and
5. Urethane Methacrylate.

Without being bound by theory, it is considered that the composition of certain embodiments contains a mix of plant oils, some of which are thought to lie adjacent to the upper surface of the nail coating and form an occlusive layer. This adsorbic effect is a result of the ionic bond which is thought to be present between the positive charge of avocado oil and the negative charge of an acrylic polymer which is typically comprised in the nail coating. The non occlusive jojoba oil (a long chain linear ester) provided in the composition is thought to penetrate the nail coating.

It is considered that this dual plant oil action may have a synergistic effect which protects the coating from the evaporation of solvents and water, prevents the penetration of oxidising agents such as UV light and replaces lost plasticizers from the nail coating.

Aptly the composition further comprises a cosmetically acceptable carrier. In certain embodiments, the cosmetically acceptable carrier is in the form of a cream. Aptly, the composition is a fluid e.g. a cream which has a consistency which enables the composition to be easily and quickly massaged into the nail coating and which does not slide or rub off the upper surface of the nail coating easily.

### DESCRIPTION OF THE FIGURES

Certain embodiments are described in more detail by way of example only with reference to the accompanying figures in which:

FIG. 1 is an SEM image of a glass slide which has been coated with Shellac® and a composition according to Example 1 has been applied on day 0;

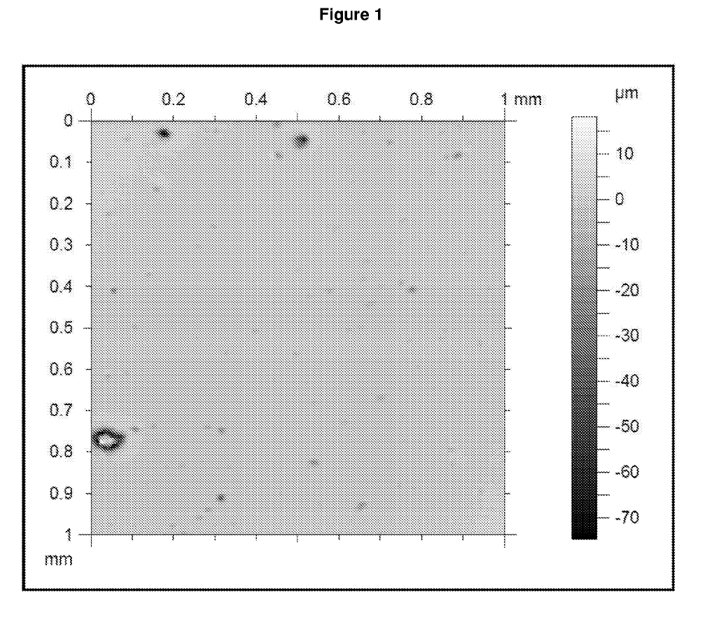

FIG. 2 is an SEM image of a glass slide which has been coated with Shellac® and a composition according to Example 1 has been applied on day 1 post-coating;

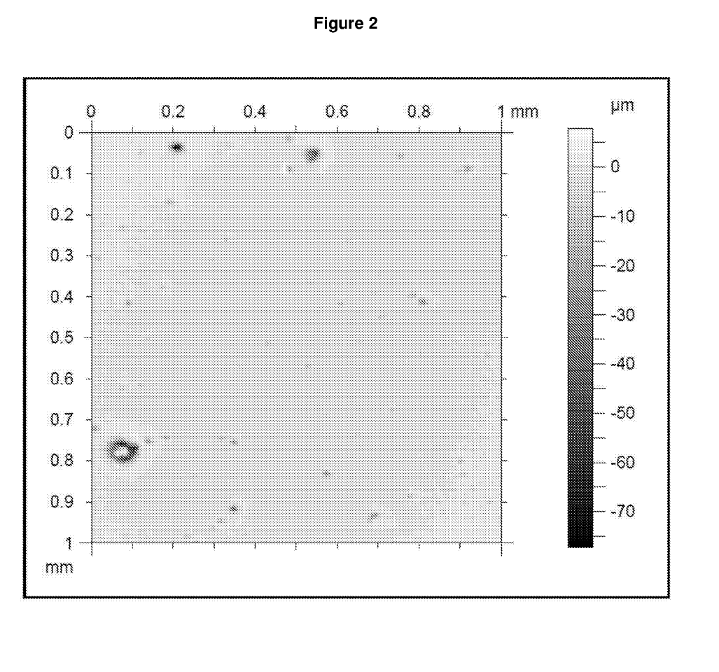

FIG. 3 is an SEM image of a glass slide which has been coated with Shellac® and a composition according to Example 1 has been applied on day 3 post-coating; and

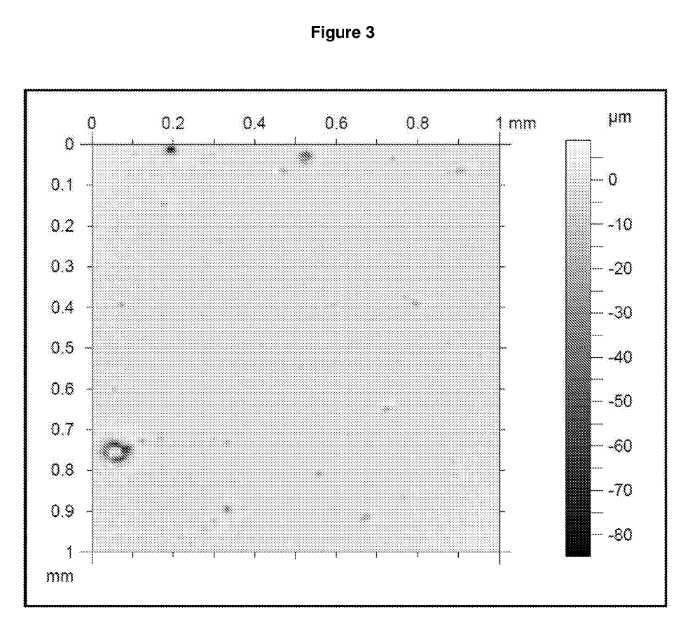

FIG. 4 is an image of a glass slide which has been coated with Shellac® and a composition according to Example 1 has been applied on day 5 post-coating.

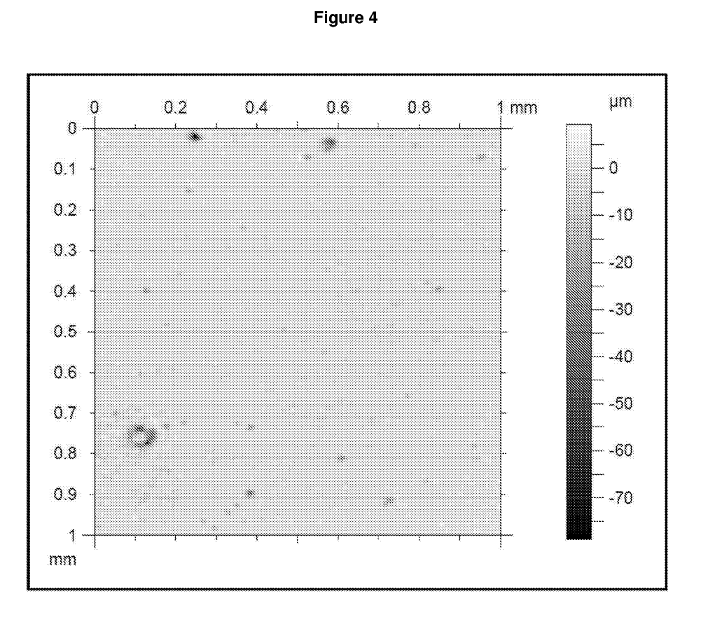

### EXAMPLES

Certain embodiments of the present invention will now be more fully illustrated using the following examples.

### Example 1

The following cream composition was prepared using the method described below.

| Material Trade Name | RM % |
|---|---|
| Deionised Water | 66.45 |
| Citric Acid Monohydrate | 0.05 |
| Versene NA2 Crystals | 0.10 |
| Surfacare ARC165 | 2.00 |
| Euxyl PE9010 | 1.10 |
| Blanova Vitamin E Acetate | 0.10 |
| Xiameter PMX-0245 | 6.00 |
| Cyclopentasiloxane | |
| Avocado Oil Refined | 0.20 |
| Lanol 99 | 8.00 |
| Jojoba Oil Refined | 3.00 |
| Montanov 202 | 3.00 |
| Ianol P | 5.00 |
| Simulgel EG | 2.50 |
| Sepimat CP 5 | 1.00 |
| Pink Pepper Pod Frag A322904 | 0.50 |
| Acqua-Biomin Copper Y3 PF | 0.50 |
| AC Colloidal Oat Flour (Avena | 0.50 |
| Sativa (Oat) Kernel | |
| Flour)*22003 | |

All concentration % are w/w.

Method of Preparation
1. Weigh out water and heat to 75° C. Then add Versene NA2 crystals and Sepimat CP5 (“aqueous phase”);
2. In a separate vessel heat Montanov 202, lanol P, Surfacare 165 ARC, Lanol 99, jojoba oil, blanova vitamin E and avocado oil (“oil phase”);
3. Add the oil phase to the aqueous phase and homogenise for 5 minutes;
4. Subsequently add Xiameter PMX 0245 and colloidal oat Flour and Euxyl PE 9010;
5. Cool to 40° C. and add with stirring Acqua-Biomin Copper Y3 PF and fragrance and ⅔ of the citric acid. Once fully mixed in then add simulgel EG with slow homogenisation. Fast homogenisation is not used in order to avoid over aeration.
6. When the batch is completely smooth and homogenous, test for pH and Viscosity.

### Example 2

### Analytical Study—the Nano-Mechanical Effect of Plant Oils on Commercially Available Nail Coatings.

The effect of a composition according to certain embodiments of the present invention on three types of commercially available nail coating was analysed.

### Protocol

A composition was prepared according to Example 1.

Samples of three nail varnish types were prepared on to a glass slide using the following methods;

### Traditional Nail Polish

Acetone cleansed with cotton wool buds and left to evaporate.
Shake polish vigorously.
Apply thin layer to slide.
Once tacky dry, repeat a second layer.
Once tacky dry apply a layer of Vinylux.

### Shellac®

Sic Paper (super fine grade #1000) used to rough the surface of the slide to help varnish adhesion.
Acetone cleansed with cotton wool buds and left to evaporate.
Shake base coat polish vigorously.
Apply thin layer of base coat and cure under UV lamp for 10 secs.
Shake colour coat polish vigorously.
Apply thin layer of colour coat and cure under UV lamp for 2 mins.
Apply thin layer of colour coat and cure under UV lamp for 2 mins.
Shake top coat polish vigorously.
Apply thin layer of top coat and cure under UV lamp for 2 mins.
Wipe top later with cotton wool buds soaked in IPA (Isopropyl Alcohol).

### Removable Gel

Sic Paper (super fine grade #1000) used to rough the surface of the slide to help varnish adhesion.
Acetone cleansed with cotton wool buds and left to evaporate.
Shake base coat polish vigorously.
Apply thin layer of base coat and cure under UV lamp for 10 secs.
Using a No6 Flat brush removable sculpting gel was applied evenly to the slide and cured under UV lamp for 2 mins.
Shake top coat polish vigorously.
Apply thin layer of top coat and cure under UV lamp for 2 mins.
Wipe top later with cotton wool buds soaked in IPA.

Samples were placed in separate petri dishes to reduce any dust/contamination coming into contact with the surface. These samples were left for 2 weeks to thoroughly set and cure before analysis.

Samples were then measured using:
a Nikon SMZ10A Stereomicroscope in co-axial light mode to highlight surface differences and by SEM imaging using a FEI Quanta FEG 250 Environmental SEM.
The profilometry work was carried out on a Taylor Hobson Talysurf CLI1000 using a CLA-3000 gauge.

The test parameters were as follows:
area scanned: 1 mm×1 mm, across the length in the centre of the specimen
grid size: 5 micron
speed: 1 mm/s
sampling rate: 200 Hz

### Sample Analysis Schedule:

Untreated imaging and surface profiles. Composition application.
Washing using di-ionised water, 24 hour imaging and surface profiles. Composition application.
Washing using di-ionised water and reapplication of composition.
Washing using di-ionised water, 72 hour imaging and surface profiles. Composition application.
Washing using di-ionised water and reapplication of composition.
Washing using di-ionised water, 120 hour surface profiles. Composition application.
Washing using di-ionised water and reapplication of composition.
Washing using di-ionised water, 168 hour surface profiles. Composition application.
Washing using di-ionised water and reapplication of composition.
Washing using di-ionised water, 216 hour surface profiles.

### Washing Method

Samples were rinsed clean using di-ionised water. As the samples were slightly waterproof, this was difficult to rinse away so samples were wetted with di-ionised water and left for a couple minutes, di-ionised water was then rinsed over the top allowing the cream to come away from the surface. It is likely that residual cream was still present on the surface before surface profile measurements.

### Observations

The composition is readily applied to the nail coatings with excess cream easy to remove without rubbing or washing.
The composition remains on the surface after washing and so is considered to have filled cracks and crevices in the nail coatings.

### Surface Profiling

The surface profiling images present an optical representation of the nail coating surfaces.

The key measurement for the surface profiling is the Sa reading which is an average surface roughness measurement across the area monitored.

Each scan was leveled to ensure compatible orientation. The surface roughness Sa (average departure from the mean plane) was then calculated for the leveled surface.

Three renderings of the data were presented:
top down pseudo photo
top down false colour image
3D axonometric view
   α=50°
   β=60°
   height amplification=10%

Another set of images was also produced with the aim of exaggerating the relief of the surface:
top down pseudo photo with light settings
   α=40
   β=40°
   contrast=8
   intensity=100
top down false colour image
3D axonometric view
   α=50°
   β=60°
   height amplification=30%

| | Traditional - um | Shellac - um | Gel - um |
|---|---|---|---|
| Day 0 | 0.303 | 0.819 | 1.24 |
| Sa 24 hours | 0.303 | 0.731 | 1.19 |
| Sa 72 hours | 0.355 | 0.806 | 3.22 |
| Sa 120 hours | 0.514 | 0.872 | 3.35 |
| Sa 168 hours | 0.493 | 0.754 | 3.17 |
| Sa 216 hours | 1.03 | 1.18 | 3.26 |

The roughness results indicate that the composition appears to interact with the surface of the coating from Day 1 with respect to the Shellac® nail coating and the gel polish and from Day 3 with respect to the nitrocellulose-based nail polish. From about day 7, there was a universal increase in the roughness of the nail coating which suggests that the nail coating has degraded by this point.

In certain embodiments, the process includes multiple applications of the inventive composition to the nail coating.

Throughout the description and claims of this specification, the words “comprise” and “contain” and variations of them mean “including but not limited to” and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any foregoing embodiments. The invention extends to any novel one, or novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A composition the composition being for application to an applied and cured nail coating, and wherein the composition comprises at least one aqueous phase and at least one oily phase, the oily phase comprising at least one wax ester and at least one plant oil component, wherein the composition is in the form of a cream and wherein the at least one plant oil component has a positive ionic charge.

2. The composition according to claim 1, wherein the composition is in the form of an oil-in-water composition.

3. The composition according to claim 2, wherein the at least one plant oil component is avocado oil or a derivative thereof.

4. The composition according to claim 1, which comprises the at least one plant oil component in a concentration of between about 0.1 and about 0.5% w/w.

5. The composition according to claim 1, wherein the at least one wax ester is a jojoba oil wax ester.

6. The composition according to claim 5, which comprises jojoba oil and/or a hydrogenated jojoba oil.

7. The composition according to claim 1, which comprises the wax ester in a concentration of between about 1% and about 3% w/w of the total weight of the composition.

8. The composition according to claim 1, wherein the aqueous phase comprises deionised water, and wherein the deionised water is provided in a concentration of between 50% and 75% w/w of the composition.

9. The composition according to claim 1, which comprises at least one emulsifier.

10. The composition according to claim 9, which comprises a plurality of emulsifiers.

11. (canceled)

12. The composition according to claim 9, wherein at least one emulsifier comprises arachidyl alcohol, behenyl alcohol & arachidyl glucoside, wherein optionally the emulsifier is Montanov 202.

13. The composition according to claim 9, wherein the at least one emulsifier comprises glyceryl stearate and PEG-100 stearate, wherein optionally the at least one emulsifier is Surfacare ARC 165.

14. (canceled)

15. The composition according to claim 9, which comprises a total concentration of emulsifiers of between about 3% and about 7% w/w, and optionally between about 4% and about 6% w/w.

16. The composition according to claim 1, which further comprises one or more of the following:
a) a perfume;
b) a texturizing agent;
c) at least one preservative; and/or
d) at least one anti-inflammatory agent.

17.-20. (canceled)

21. The composition according to claim 1, wherein the composition comprises a metal component selected from a silver-containing component, a zinc containing component and a copper containing component.

22. (canceled)

23. The composition according to claim 22, wherein the copper containing component is a copper complex.

24. The composition according to claim 21, which comprises a plurality of anti-inflammatory agents.

25. The composition according to claim 21, which further comprises colloidal oat flour.

26. The composition according to claim 1, which further comprises a chelating agent, wherein optionally the chelating agent is EDTA or a salt thereof.

27. The composition according to claim 1, which further comprises at least one emollient.
